(19) United States
(12) Patent Application Publication
(10) Pub. No.: US 2013/0296642 A1
(43) Pub. Date: Nov. 7, 2013

(54) BIOMARKERS AND METHOD FOR PREDICTING OCCURENCE OF VENTRAL HERNIAS

(71) Applicant: THE CURATORS OF THE UNIVERSITY OF, Columbia, MO (US)

(72) Inventors: Ulus Atasoy, Columbia, MO (US); Sharon L. Bachman, Columbia, MO (US); Robert Calaluce, Columbia, MO (US); Wade Davis, Columbia, MO (US); Bruce J. Ramshaw, Columbia, MO (US)

(21) Appl. No.: 13/827,472

(22) Filed: Mar. 14, 2013

Related U.S. Application Data
(60) Provisional application No. 61/685,761, filed on Mar. 24, 2012.

(51) Int. Cl. A61F 2/00; C12Q 1/68

(52) U.S. Cl. CPC A61F 2/0063; C12Q 1/6883
USPC 600/37; 506/9; 435/7.9; 506/17

(57) ABSTRACT

Kits, methods of treating, and methods of diagnosing a risk level for an incisional hernia in a subject undergoing abdominal surgery are disclosed. They are designed to determine risk factors for incisional hernia formation based on a subject's unique gene expression profiles.

### CROSS-REFERENCES TO RELATED APPLICATIONS

This application claims the benefit of U.S. Provisional application No. 61/685,761 filed on Mar. 24, 2012, each of which is herein incorporated in its entirety by reference.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND OF THE INVENTION

1. Field of the Invention

The present invention relates to methods of predicting the occurrence of ventral hernias before initial surgery, and to methods of treating abdominal surgical incisions.

2. Description of Related Art

Incisional hernia repair comprises a significant proportion of a general surgeon's practice. The incidence of incisional hernias ranges from 2% to 11%, with a substantial recurrence rate reported between 10% and 50%. Based upon this estimate, 100,000 incisional hernia repairs are predicted to be performed each year costing $2.5 billion. While recurrence rates have decreased by using prosthetic mesh in the repair, a significant number of patients develop multiple recurrences with estimates in the literature ranging from 5% to 20%.

Several risk factors for developing incisional hernias have been identified including wound infection, abdominal distention, pulmonary complications, male gender, age, and obesity. Although risk factors for recurrent incisional hernias have also been evaluated, the literature is controversial with regard to many of these, such as body mass index, ascites, large hernias exceeding 10 cm in width or length, continued smoking, occupational lifting, and wound healing disorders (e.g., hematoma, seroma, infection).

Current data suggests that incisional hernias are commonly caused by failure of early surgical wound healing. Since collagen I provides tensile strength to connective tissue, and immature collagen III found in early wounds is weaker, investigations of the collagen I to III ratio have demonstrated a decreased ratio in patients with direct and indirect hernias as compared with controls. This decrease in the collagen I/III ratio was attributed to the relative increase in collagen III synthesis and was seen in incisional hernias. Moreover, a decreased collagen I/III ratio in incisional hernias supports the possibility of a high-risk group more susceptible to hernia formation. White and colleagues performed a preliminary immunohistochemical trial examining the skin and fascia of 16 incisional hernia patients for collagen I and III and compared the ratio to normal foregut collected from bariatric patients. They found a significant decrease in the ratio in the skin of the hernia patients but found no difference in the fascia. While patients with collagen and connective tissue diseases, such as Ehlers-Danlos, osteogenesis imperfecta, and Marfan's syndromes, are known to form hernias, there is no data on potential genetic predispositions to hernia formation in otherwise normal patients.

Thus, there remains a need for improved methods for diagnosing and predicting the risk of hernia occurrence of a particular patient before initial surgery, which provides guidance on treatment and incision healing for a particular patient.

### BRIEF SUMMARY OF THE INVENTION

Accordingly, the inventors herein disclose new methods for determining a risk level for an incisional hernia in a subject following surgery, and methods for treating the subject based upon the subject's risk level. The methods are designed to provide targeted patient specific therapy.

Thus, in various embodiments, the present invention provides a method of treating an abdominal surgical incision in a subject in need thereof comprising the steps of: (a) measuring the level of expression of a gene or combination of genes selected from the group consisting of COL1A2, COL3A1, GREM1, and IL10 in a tissue sample obtained from the subject; (b) comparing the level of expression of the gene or combination of genes in the tissue sample to that in a control sample; (c) determining a risk level for an incisional hernia in the subject following surgery, wherein the subject has a high risk for an incisional hernia if the level of expression of the gene or combination of genes in the subject's tissue sample displays a greater than about 1.5 fold change compared to the level of expression of the gene or combination of genes in the control sample, and a normal risk for an incisional hernia if the level of expression of the gene or combination of genes in the subject's tissue sample displays a about 1.5 fold change or less compared to the level of expression of the gene or combination of genes in the control sample; and (d) treating the subject based upon the subject's risk level for an incisional hernia as determined in step c. In certain embodiments, the gene is GREM1.

In certain embodiments, the tissue sample is obtained from the subject's skin, blood, or fascia.

In certain embodiments, the subject has a high risk for an incisional hernia if the level of expression of GREM1 in the subject's tissue sample is lower than the level of expression of GREM1 in the control sample. In certain embodiments, the subject has a high risk for an incisional hernia if the level of expression of COL1A2, COL3A1, or IL10, or combinations thereof, in the subject's tissue sample is higher than that in the control sample.

In various embodiments, the level of gene expression is measured by microarray, PCR array, or immunohistochemistry. In certain embodiments, the level of gene expression is measured via the quantity of nucleic acid transcripts produced by the gene or combination of genes. In certain embodiments, the gene expression is measured via the quantity of protein expressed from the gene or combination of genes.

In various embodiments, the subject determined to have a high risk for an incisional hernia is treated with placement of surgical mesh in the abdominal incision. In various embodiments, the surgical mesh is affixed to abdominal tissue bridging the opening in the abdomen. In various embodiments, the subject determined to have a high risk for an incisional hernia is treated with laparoscopic surgery.

In various embodiments, the present invention provides a method of diagnosing a risk level for an incisional hernia in a subject following surgery, comprising the steps of: (a) measuring the level of expression of a gene or combination of genes selected from the group consisting of COL1A2, COL3A1, GREM1, and IL10 in a tissue sample obtained from the subject; (b) comparing the level of expression of the gene or combination of genes in the tissue sample to that in a control sample; (c) determining a risk level for an incisional hernia in the subject following surgery, wherein the subject has a high risk for an incisional hernia if the level of expression of the gene or combination of genes in the subject's tissue sample displays a greater than about 1.5 fold change compared to the level of expression of the gene or combination of genes in the control sample, and a normal risk for an incisional hernia if the level of expression of the gene or combination of genes in the subject's tissue sample displays a about 1.5 fold change or less compared to the level of expression of the gene or combination of genes in the control sample. In certain embodiments, the gene is GREM1.

In various embodiments, the tissue sample is obtained from the subject's skin, blood, or fascia.

In certain embodiments, the subject has a high risk for an incisional hernia if the level of expression of GREM1 in the subject's tissue sample is lower than the level of expression of GREM1 in the control sample. In certain embodiments, the subject has a high risk for an incisional hernia if the level of expression of COL1A2, COL3A1, or IL10, or combinations thereof, in the subject's tissue sample is higher than that in the control sample.

In various embodiments, the gene expression is measured by microarray, PCR array, and/or immunohistochemistry. In certain embodiments, the level of gene expression is measured via the quantity of nucleic acid transcripts produced by the gene or combination of genes. In certain embodiments, the gene expression is measured via the quantity of protein expressed from the gene or combination of genes.

In various embodiments, the present invention provides a product comprising isolated biomarkers bound to a biochip array, wherein the biomarkers are selected from COL1A2, COL3A1, GREM1, IL10, or combinations thereof. In a particular embodiment, the biomarker is GREM1.

In various embodiments, the present invention provides a product comprising purified biomarkers bound to a microarray comprising addressable locations using a biospecific capture reagent, wherein the biomarkers are selected from COL1A2, COL3A1, GREM1, IL10, or combinations thereof. In a particular embodiment, the biomarker is GREM1.

In various embodiments, the present invention provides a kit for diagnosing a risk level for an incisional hernia in a subject following surgery, comprising: (a) purified biomarkers bound to a microarray comprising addressable locations using an adsorbent or capture reagent, wherein the purified biomarkers are selected from COL1A2, COL3A1, GREM1, IL10, or combinations thereof, and (b) written instructions for diagnosing a risk level for an incisional hernia in a subject following abdominal surgery, comprising the steps of: (i) measuring the level of expression of a gene or combination of genes selected from the group consisting of COL1A2, COL3A1, GREM1, and IL10 in a tissue sample obtained from the subject; (ii) comparing the level of expression of the gene or combination of genes in the tissue sample to that in a control sample; (iii) determining a risk level for an incisional hernia in the subject following surgery, wherein the subject has a high risk for an incisional hernia if the level of expression of the gene or combination of genes in the subject's tissue sample displays a greater than about 1.5 fold change compared to the level of expression of the gene or combination of genes in the control sample, and a normal risk for an incisional hernia if the level of expression of the gene or combination of genes in the subject's tissue sample displays a about 1.5 fold change or less compared to the level of expression of the gene or combination of genes in the control sample. In a particular embodiment, the biomarker is GREM1.

In various embodiments, the present invention provides a biochip array of isolated biomarkers, wherein the biomarkers are COL1A2, COL3A1, GREM1, IL10, or combinations thereof. In a particular embodiment, the biomarker is GREM1.

In various embodiments, the present invention provides a biochip array having a plurality of addressable locations, each comprising at least one isolated biomarker, wherein the biomarkers are selected from the group consisting of COL1A2, COL3A1, GREM1, IL10, and combinations thereof.

In a particular embodiment, the biomarker is GREM1.

In various embodiments, the biochip array has at least two addressable locations, each comprising at least one isolated biomarker, wherein the two or more biomarkers are selected from the group consisting of COL1A2, COL3A1, GREM1, IL10, and combinations thereof. In various embodiments, the biochip array has at least three addressable locations, each comprising at least one isolated biomarker, wherein the three or more biomarkers are selected from the group consisting of COL1A2, COL3A1, GREM1, IL10, and combinations thereof. In various embodiments, the biochip array has at least four addressable locations, each comprising at least one isolated biomarker, wherein the four biomarkers are COL1A2, COL3A1, GREM1, and IL10.

In various embodiments, the present invention provides a product comprising at least one isolated biomarker bound to a bead by a biospecific capture reagent, wherein the biomarkers are selected from the group consisting of COL1A2, COL3A1, GREM1, IL10, and combinations thereof. In various embodiments, one biomarker is bound to the bead. In a particular embodiment, the biomarker is GREM1. In a particular embodiment, the biospecific capture reagent is an antibody.

In various embodiments, the product comprises at least two bead types. In various embodiments, the product comprises at least three bead types. In various embodiments, the product comprises at least four bead types. In a particular embodiment, at least one of the biomarkers is GREM1. In particular embodiments, the biospecific capture reagent is an antibody.

In various embodiments, the product comprises at least two isolated biomarkers bound to a bead by a biospecific capture reagent, wherein the biomarkers are selected from the group consisting of COL1A2, COL3A1, GREM1, IL10, and combinations thereof. In a particular embodiment, at least one of the biomarkers is GREM1. In particular embodiments, the biospecific capture reagent is an antibody.

In various embodiments, the present invention provides a biochip comprising one or more isolated biomarkers, wherein the biomarkers are selected from the group consisting of COL1A2, COL3A1, GREM1, IL10, or combinations thereof. In a particular embodiment, the biomarker is GREM1. In a particular embodiment, the isolated biomarkers are present at addressable locations.

In various embodiments, the biochip has at least two addressable locations, each comprising a different isolated biomarker, wherein the biomarkers are selected from the group consisting of COL1A2, COL3A1, GREM1, IL10, and combinations thereof. In various embodiments, the biochip has at least three addressable locations, each comprising a different isolated biomarker, wherein the biomarkers are selected from the group consisting of COL1A2, COL3A1, GREM1, IL10, and combinations thereof. In various embodiments, the biochip has at least four addressable locations, each comprising a different isolated biomarker, wherein the biomarkers are COL1A2, COL3A1, GREM1, and IL10.

### BRIEF DESCRIPTION OF THE DRAWINGS

FIG. 1 shows agreement of microarray and PCR array results. The genes that were detected on both the microarray and the PCR array are plotted against their fold change (RH/NC) for each platform. Bold italicized gene symbols indicate they were significantly different based on microarray data; and

FIG. 2 shows patient-level gene expression data for four selected genes from PCR array with group median indicated by a horizontal line; and

FIG. 3 shows the ability of the combination of GREM1 and COL3A1 gene expression to separate recurrent hernia (RH) and normal control (NC) patients. PCR array data were used to explore the utility of gene expression of GREM1 and COL3A1 as markers to distinguish RH and NC patients. The best separation boundary (solid line) was determined using quadratic discriminant analysis. Using all of the data, only 1 patient (RH, gray) was misclassified (93% accuracy). Using leave-one-out cross-validation, in which each patient's data is held out (in turn) during the calculation of the best boundary and subsequently evaluated for accuracy, 13/15 (86%) patients were correctly classified.

### DETAILED DESCRIPTION OF THE INVENTION

### Abbreviations and Definitions

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein when referring to a measurable value such as an amount of a compound, dose, time, temperature, and the like, is meant to encompass variations of 20%, 10%, 5%, 1%, 0.5%, or even 0.1% from the specified amount.

The term “gene” as used herein to describe a discrete nucleic acid locus, unit or region within a genome that may comprise one or more of introns, exons, splice sites, open reading frames and 5′ and/or 3′ non-coding regulatory sequences such as a promoter and/or a polyadenylation sequence. “Gene” also encompasses an RNA copy or cDNA copy of the gene.

The term “expression” refers to the transcription of a gene to produce the corresponding mRNA and translation of this mRNA to produce the corresponding gene product (i.e., a peptide, polypeptide, or protein).

The term “biomarker” typically refers to a protein, found in a tissue sample, whose level varies and may be readily quantified. The quantified level may then be compared to a known value. The comparison may be used for several different purposes, including but not limited to, prognosis of incisional hernia risk, and treatment of abdominal surgical wounds.

Biomarkers are detected by any methodology that can detect and distinguish the biomarker. A method for detection involves first capturing the biomarker and modified forms of it, e.g., with biospecific capture reagents, and then detecting the captured proteins by mass spectrometry. More specifically, the proteins are captured using biospecific capture reagents, such as antibodies, aptamers or Affibodies that recognize the biomarker. This method also will also result in the capture of protein interactors that are bound to the proteins or that are otherwise recognized by antibodies and that, themselves, can be biomarkers. Preferably, the biospecific capture reagents are bound to a solid phase. Then, the captured proteins can be detected by mass spectrometry or by eluting the proteins from the capture reagent and detecting the eluted proteins by mass spectrometry.

The term “microarray” is used interchangeably with “array,” “gene chip,” “DNA chip,” “biochip,” and refers to a plurality of spots of oligonucleotides on a solid support for use in probing a biological sample to determine gene expression, marker pattern or nucleotide sequence. Examples of supports include, but are not limited to, glass, silica chips, nylon (polyamide) membrane, polymer, plastic, ceramic, metal, coated on optical fibers, or infused into a gel matrix.

Preferably, the biospecific capture reagent is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies, such as xMAP technology of Luminex (Austin, Tex.) can be used to detect the protein clusters. However, the biospecific capture reagents must be specifically directed toward the members of a cluster in order to differentiate them.

In yet another embodiment, the surfaces of biochips can be derivatized with the capture reagents directed against protein clusters either in the same location or in physically different addressable locations. One advantage of capturing different clusters in different addressable locations is that the analysis becomes simpler.

The term “immunohistochemistry” refers to the process of detecting antigens in cells of a tissue section or cell sample based on the principle of antibodies binding specifically to antigens in biological tissues. IHC has become a major tool to analyze the existence, localization and distribution of proteins of interest and is therefore widely used for diagnostic purposes. Generally, during an IHC analysis, a tissue section or cell sample is fixed on the surface of a glass slide and then submitted to immunostaining with antigen specific antibodies.

### Methods

The present invention provides new methods for determining a risk level for an incisional hernia in a subject following abdominal surgery, and methods for treating the subject based upon the subject's risk level. Without wishing to be bound by any particular theory, gene expression profiles may act as surrogate markers that stratify patients into different groups at risk for hernia development prior to their initial surgery.

Microarray experiments revealed distinct differences in the gene expression profiles between patients presenting for recurrent incisional hernia and normal control patients. One hundred and sixty-seven genes in the skin and seven genes in the fascia were differentially expressed, including eight directly involved in collagen synthesis. In particular, GREMLIN1, or bone morphogenetic protein antagonist 1, was under expressed in skin (fold=0.49, p<10⁻⁷, q=0.0009) and fascia (fold=0.23, p<10⁻⁴, q=0.095) of recurrent incisional hernia patients compared with normal control. The PCR array data supported previous reports of decreased collagen I/III ratios in skin of recurrent incisional hernia versus normal control (mean=1.51±0.73 vs. mean=2.26±0.99; one-sided t test, p=0.058).

Thus, in various embodiments, the present invention provides a method of treating an abdominal surgical incision in a subject in need thereof comprising the steps of: (a) measuring the level of expression of a gene or combination of genes selected from the group consisting of COL1A2, COL3A1, GREM1, and IL10 in a tissue sample obtained from the subject; (b) comparing the level of expression of the gene or combination of genes in the tissue sample to that in a control sample; (c) determining a risk level for an incisional hernia in the subject following surgery, wherein the subject has a high risk for an incisional hernia if the level of expression of the gene or combination of genes in the subject's tissue sample displays a greater than about 1.5 fold change compared to the level of expression of the gene or combination of genes in the control sample, and a normal risk for an incisional hernia if the level of expression of the gene or combination of genes in the subject's tissue sample displays a about 1.5 fold change or less compared to the level of expression of the gene or combination of genes in the control sample; and (d) treating the subject based upon the subject's risk level for an incisional hernia as determined in step c. In certain embodiments, the gene is GREM1. In certain embodiments, the tissue sample is obtained from the subject's skin, blood, or fascia.

The present invention includes methods that quantify expression levels in clinical samples as well as methods that determine whether a gene of interest is expressed at all or expressed above a threshold (e.g., a control threshold) in clinical samples. Thus, an assay that provides a “yes or no” result without necessarily providing quantification of gene expression is within the scope of the present invention. The invention may involve quantitative or qualitative assessment of gene expression.

The genes identified as being differentially expressed for recurrent incisional hernia risk are used in a variety of nucleic acid detection assays to detect or quantify the expression level of a gene or multiple genes in a given sample. Examples include, but are not limited to traditional Northern blotting, nuclease protection, RT-PCR and differential display methods may be used for detecting gene expression levels, including Taqman and flap endonuclease assays. Additional assays include array or chip hybridization-based methods, which are convenient when determining the expression levels of a larger number of genes.

As used herein, the term “control” refers to a specific value or dataset that can be used to prognose or classify the value e.g expression level or reference expression profile obtained from the test sample associated with an outcome class. In one embodiment, a dataset may be obtained from samples from a group of subjects known to have recurrent incisional hernias. In another, a dataset may be obtained from samples from a group of subjects known to have no risk of recurrent incisional hernias. The expression data of the biomarkers in the dataset can be used to create a “control value” that is used in testing samples from new patients. A control value is obtained from the historical expression data for a patient or pool of patients with a known outcome. In some embodiments, the control value is a numerical threshold for predicting outcomes, for example good and poor outcome, or making therapy recommendations, for laparoscopic surgery instead of open surgery.

In some embodiments, the “control” is a predetermined value for the set of biomarkers obtained from patients whose biomarker expression values and hernia risk are known.

In various embodiments, the sample from the subject is one or more of blood, blood plasma, serum, urine, cells, organs, seminal fluids, bone marrow, saliva, stool, a cellular extract, or cerebrospinal fluid. Certain embodiments, the tissue sample is obtained from the subject's skin or fascia. In certain embodiments, the tissue sample is obtained from the subject's blood. Those of skill in the art will know of other samples well suited for use in the present invention.

In certain embodiments, the subject has a high risk for an incisional hernia if the level of expression of GREM1 in the subject's tissue sample is lower than the level of expression of GREM1 in the control sample. In certain embodiments, the subject has a high risk for an incisional hernia if the level of expression of COL1A2, COL3A1, or IL10, or combinations thereof, in the subject's tissue sample is higher than that in the control sample.

In various embodiments, the level of gene expression is measured by microarray, PCR array, or immunohistochemistry. In certain embodiments, the level of gene expression is measured via the quantity of nucleic acid transcripts produced by the gene or combination of genes. In certain embodiments, the gene expression is measured via the quantity of protein expressed from the gene or combination of genes.

As used herein, the term “differential expression” refers to a difference in the level of expression of the products of one or more biomarkers. For instance, the term “differential expression” can refer to the difference in the level of RNA of one or more biomarkers between samples from subjects having and subjects not having a risk for incisional hernias. Differences in biomarker RNA product levels can be determined by directly or indirectly measuring the amount or level of RNA or protein. “Differentially expressed” can also include different levels of protein encoded by the biomarker of the invention between samples or reference populations. “Differential expression can be determined as the ratio of the levels of one or more biomarker products between reference subjects/populations having or not having a risk for incisional hernias, wherein the ratio is not equal to 1.0. Differential expression between populations can be determined to be statistically significant as a function of p-value. When using p-value to determine statistical significance, a biomarker, the p-value is preferably less than 0.2. In another embodiment the biomarker is identified as being differentially expressed when the p-value is less than 0.15, 0.1, 0.05, 0.01, 0.005, 0.0001 etc. When determining differential expression on the basis of the ratio, a biomarker product is differentially expressed if the ratio of the level of expression in a first sample as compared with a second sample is greater than or less than 1.0. For example, a ratio of greater than 1.0 for example includes a ratio of greater than 1.1, 1.2, 1.5, 1.7, 2, 3, 4, 10, 20 and the like. A ratio of less than 1.0, for example, includes a ratio of less than 0.9, 0.8, 0.6, 0.4, 0.2, 0.1, 0.05 and the like. In another embodiment of the invention a biomarker product is differentially expressed if the ratio of the mean of the level of expression of a first population as compared with the mean level of expression of the second population is greater than or less than 1.0. For example, a ratio of greater than 1.0 includes a ratio of greater than 1.1, 1.2, 1.5, 1.7, 2, 3, 4, 10, 20 and the like and a ratio less than 1.0, for example includes a ration of less than 0.9, 0.8, 0.6, 0.4, 0.2, 0.1, 0.05 and the like. In another embodiment of the invention a biomarker product is differentially expressed if the ratio of its level of expression in a first sample as compared with the mean of the second population is greater than or less than 1.0 and includes for example, a ratio of greater than 1.1, 1.2, 1.5, 1.7, 2, 3, 4, 10, 20, or a ratio less than 1, for example 0.9, 0.8, 0.6, 0.4, 0.2, 0.1, 0.05.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold higher or more, than a control.

“Differentially decreased expression” or “down regulation” refers to biomarker product levels which are at least 10% or more, for example, 20%, 30%, 40%, or 50%, 60%, 70%, 80%, 90% lower or less, and/or 0.9 fold, 0.8 fold, 0.6 fold, 0.4 fold, 0.2 fold, 0.1 fold or less lower than a control.

For example, up regulated genes include genes having an increased level of biomarker products in a test sample as compared with a control sample.

In various embodiments, the subject determined to have a high risk for an incisional hernia is treated with placement of surgical mesh in the abdominal incision. The likelihood of incisional hernias may be reduced by the placement of mesh reinforcement, such as polypropylene onlay technique or by a Gore-Tex Duramesh laparoscopic method. In general, the advantages of using prosthetic materials include availability, absence of donor site morbidity, and added strength of the prosthetic material. Bioprosthetic materials may be used as well. Three types of bioprosthetics used in current practice include acellular human dermis, porcine small intestinal submucosa (SIS), and acellular porcine dermis.

In various embodiments, the surgical mesh is affixed to abdominal tissue bridging the opening in the abdomen. In various embodiments, the subject determined to have a high risk for an incisional hernia is treated with laparoscopic surgery. The material is affixed via staples or sutures. Those of skill in the art will know of other techniques and materials well suited for use in the present invention, including surgical techniques for “tension-free repair” or a repair under “physiologic tension”, procedures designed to minimize incisional hernia formation.

In various embodiments, the present invention provides a method of diagnosing a risk level for an incisional hernia in a subject following surgery, comprising the steps of: (a) measuring the level of expression of a gene or combination of genes selected from the group consisting of COL1A2, COL3A1, GREM1, and IL10 in a tissue sample obtained from the subject; (b) comparing the level of expression of the gene or combination of genes in the tissue sample to that in a control sample; (c) determining a risk level for an incisional hernia in the subject following surgery, wherein the subject has a high risk for an incisional hernia if the level of expression of the gene or combination of genes in the subject's tissue sample displays a greater than about 1.5 fold change compared to the level of expression of the gene or combination of genes in the control sample, and a normal risk for an incisional hernia if the level of expression of the gene or combination of genes in the subject's tissue sample displays a about 1.5 fold change or less compared to the level of expression of the gene or combination of genes in the control sample. In certain embodiments, the gene is GREM1.

In various embodiments, the tissue sample is obtained from the subject's skin, blood, or fascia.

In certain embodiments, the subject has a high risk for an incisional hernia if the level of expression of GREM1 in the subject's tissue sample is lower than the level of expression of GREM1 in the control sample. In certain embodiments, the subject has a high risk for an incisional hernia if the level of expression of COL1A2, COL3A1, or IL10, or combinations thereof, in the subject's tissue sample is higher than that in the control sample.

In various embodiments, the gene expression is measured by microarray, PCR array, and/or immunohistochemistry. In certain embodiments, the level of gene expression is measured via the quantity of nucleic acid transcripts produced by the gene or combination of genes. In certain embodiments, the gene expression is measured via the quantity of protein expressed from the gene or combination of genes.

In various embodiments, gene expression is measured by microarray, PCR array, and/or immunohistochemistry.

### Kits

In one aspect, the invention provides kits for the prognosis of a risk level for an incisional hernia in the subject following abdominal surgery. The kits include PCR primers for at least one marker selected from COL1A2, COL3A1, GREM1, IL10, or combinations thereof. In preferred embodiments, the kit includes the markers GREM1. The kit may further include instructions for use and correlation of the maker with risk level. The kit may also include a DNA array containing the complement of one or more of the markers selected from COL1A2, COL3A1, GREM1, IL10, or combinations thereof, reagents, and/or enzymes for amplifying or isolating sample DNA. The kits may include reagents for real-time PCR, for example, TaqMan probes and/or primers, and enzymes.

In yet another aspect, the invention provides kits for qualifying a risk level for an incisional hernia, wherein the kits can be used to detect the markers of the present invention. For example, the kits can be used to detect any one or more of the markers described herein, which markers are differentially present in samples of normal subjects and RH subjects.

In one embodiment, a kit comprises: (a) a substrate comprising an adsorbent thereon, wherein the adsorbent is suitable for binding a marker, and (b) instructions to detect the marker or markers by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry.

Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe, which is removably insertable into a gas phase ion spectrometer. In other embodiments, the kit may comprise a single substrate, which is in the form of a removably insertable probe with adsorbents on the substrate. In yet another embodiment, the kit may further comprise a pre-fractionation spin column (e.g., Cibacron blue agarose column, anti-HSA agarose column, K-30 size exclusion column, Q-anion exchange spin column, single stranded DNA column, lectin column, etc.).

In another embodiment, a kit comprises (a) an antibody that specifically binds to a biomarker; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

Optionally, the kit may further comprise a standard or control information so that the test sample can be compared with the control information standard to determine if the test amount of a marker detected in a sample is a diagnostic amount consistent with a prognosis of an elevated risk level for an incisional hernia.

In various embodiments, the present invention provides a product comprising purified biomarkers bound to a microarray comprising addressable locations using a biospecific capture reagent, wherein the biomarkers are selected from COL1A2, COL3A1, GREM1, IL10, or combinations thereof. In a particular embodiment, the biomarker is GREM1.

In various embodiments, the present invention provides a kit for diagnosing a risk level for an incisional hernia in a subject following surgery, comprising: (a) purified biomarkers bound to a microarray comprising addressable locations using an adsorbent or capture reagent, wherein the purified biomarkers are selected from COL1A2, COL3A1, GREM1, IL10, or combinations thereof, and (b) written instructions for diagnosing a risk level for an incisional hernia in a subject following abdominal surgery, comprising the steps of: (i) measuring the level of expression of a gene or combination of genes selected from the group consisting of COL1A2, COL3A1, GREM1, and IL10 in a tissue sample obtained from the subject; (ii) comparing the level of expression of the gene or combination of genes in the tissue sample to that in a control sample; (iii) determining a risk level for an incisional hernia in the subject following surgery, wherein the subject has a high risk for an incisional hernia if the level of expression of the gene or combination of genes in the subject's tissue sample displays a greater than about 1.5 fold change compared to the level of expression of the gene or combination of genes in the control sample, and a normal risk for an incisional hernia if the level of expression of the gene or combination of genes in the subject's tissue sample displays a about 1.5 fold change or less compared to the level of expression of the gene or combination of genes in the control sample. In a particular embodiment, the biomarker is GREM1.

In various embodiments, the present invention provides a biochip array of isolated biomarkers, wherein the biomarkers are COL1A2, COL3A1, GREM1, IL10, or combinations thereof. In a particular embodiment, the biomarker is GREM1.

In various embodiments, the present invention provides a biochip array having addressable locations comprising isolated biomarkers, wherein the biomarkers are COL1A2, COL3A1, GREM1, IL10, or combinations thereof. In a particular embodiment, the biomarker is GREM1.

In various embodiments, the present invention provides a product comprising isolated biomarkers bound to a bead by a biospecific capture reagent, wherein the biomarkers are COL1A2, COL3A1, GREM1, IL10, or combinations thereof. In a particular embodiment, the biomarker is GREM1. In a particular embodiment, the biospecific capture reagent is an antibody.

In various embodiments, the present invention provides a biochip comprising isolated biomarkers, wherein the biomarkers are COL1A2, COL3A1, GREM1, IL10, or combinations thereof. In a particular embodiment, the biomarker is GREM1. In a particular embodiment, the isolated biomarkers are present at addressable locations.

In order that the invention described herein may be more fully understood, the following examples are set forth. It should be understood that these examples are for illustrative purposes only and are not to be construed as limiting this invention in any manner.

### EXAMPLES

### Patient Samples and Tissue Acquisition

Thirty-three patients participated in this study. Patients were eligible if they were 18 years of age or older and underwent laparoscopic repair of a recurrent ventral or incisional hernia. Patients were excluded if they were under 18; had a history of steroid use, severe COPD, pulmonary, or connective tissue disorders; or were prisoners. Eighteen patients with at least one recurrent incisional hernia presented for laparoscopic incisional hernia repair. The designated controls were 15 healthy patients who had no hernia history and underwent laparoscopic cholecystectomy. Approximately 1 cm² of skin and fascia was removed from the trocar placement site, remote from the hernia or old incisions. The tissue samples were divided and placed in either 10% buffered formalin or RNALater™ RNA Stabilization Reagent (Qiagen, Valencia, Calif.). Tissue was stored in RNALater™ for up to 48 h at room temperature. Approximately 100-150 mg of tissue was used for RNA isolation.

### RNA Isolation and RNA Amplification

Total RNA was isolated from the skin and fascia specimens by following the manufacturer's protocol from the RNeasy® Lipid Tissue Mini Kit (Qiagen) using a rotor homogenizer and on-column DNase treatment. Total RNA was amplified using the WT-Ovation™ Pico RNA Amplification System protocol (NuGen, San Carlos, Calif.) as previously described [12, 13].

cDNA Labeling, RNA Quantity and Quality, and Microarray

Of the 33 enrolled patients, 8 normal control (NC) and 9 recurrent incisional hernia (RH) patients were selected for microarray analysis based on the quantity, quality, and integrity of the RNA. For each skin and fascia sample, 1.5 μg biotin labeled, amplified cDNA was hybridized to a Sentrix® Human-6 v.2 Whole Genome Expession BeadChips (Sentrix Human WG-6; Illumina, San Diego, Calif.) as previously described [13].

Validation by Quantitative RT-PCR (qPCR) and PCR Array

cDNA was generated from 10 ng of the same total RNA samples as used for the microarray experiment (15 patients analyzed by microarray with sufficient amounts of remaining high-quality RNA) and SuperScript™ III Platinum® Two-Step qPCR Kit with SYBR® Green (Invitrogen Carlsbad, Calif.). For COL1A and GREM1, qPCR was performed on the StepOne™ Real-Time PCR System (Applied Biosystems, Foster City, Calif.) using GAPDH as a reference gene as previously described [13]. A PCR array, focusing on the expression of 84 key genes related to dysregulated tissue remodeling during wound healing, was also performed on these 15 patients by Global Biologics (Columbia, Mo.). Briefly, RNA quantity and purity were assessed using NanoDrop ND-2000 (Nanodrop Technologies, Wilmington, Del., USA). RNA integrity was evaluated using the RNA integrity algorithm generated by the Bioanalyzer 2100 with the Eukaryotic RNA Pico Series II reagents (Agilent Technologies, Santa Clara, Calif., USA). RIN values ranged from 5 to 8. RNA was reverse transcribed with the RT2 First Strand cDNA kit (SABiosciences, Frederick, Md.), and qPCR was performed using the Human Fibrosis RT2 Profiler™ PCR Array System (SABiosciences, Frederick, Md.) and the Roche LightCycler480 instrument. As part of the qPCR quality assessment process, each sample was evaluated for the presence of genomic cDNA contamination, followed by three positive PCR and three reverse transcriptase controls. The chosen housekeeping or reference gene, RPL13A, was selected from a panel of five housekeeping genes on the array based on the most uniform expression range across all samples. GREM1 and COL1A qPCR data were statistically compared using a two-sample t test on the ΔCt values. The PCR array data were compared between groups using a moderated t test on the ΔCt values as long as the gene was considered to be reliably expressed (Ct<35 in 75% of samples) [14].

### Immunohistochemistry

Specimens were fixed in 10% buffered formalin, routinely processed, embedded in paraffin, and cut at 4 μm. Immunohistochemistry was performed using the automated horseradish peroxidase Autostainer/Envision Plus method (Dakocytomation, Carpenteria, Calif.) as previously described [15, 16].

### Statistical Analysis of Microarray Data

Analysis of microarray gene expression data was primarily performed using R open-source software (R Foundation, Vienna, Austria). Any genes considered “not detectable” (Illumina software detection <1%) across >50% of patient samples were excluded from further statistical analyses in order to reduce false positives. Nonspecific filtering was also carried out to remove genes with little variability as previously described [17]. Differential gene expression analysis was performed using a moderated t statistics applied to the log 2-transformed normalized intensity for each gene using an empirical Bayes approach [14]. Adjustment for multiple testing was made using the false discovery rate method of Benjamini and Hochberg with a significance cutoff of q<30% [18], since the list of discovered genes was relatively small. We declared a gene differentially expressed if it was statistically significant after adjusting for multiple testing and had a fold change ≧1.5 (either over- or under expressed).

Gene ontology (GO) analyses were conducted on the resulting list of significantly different genes to test their association with independently established GO terms to shed insight on the common functions of the differentially identified genes. We carried out GO analyses for overrepresentation of biologic process, molecular function, and cellular component ontologies, which generated an odds ratio (OR) and p value for each GO category, using methods previously described [13]. A small p value (<0.05) and large OR indicated that the number of selected genes associated with a given term (e.g., wound healing) was larger than expected due to chance. GO categories containing less than 10 genes represented on the array were not considered to be statistically reliable indicators and were not reported even if significant.

### Demographics

Demographics for the 33 enrolled patients and the subset of 17 patients whose samples were analyzed by microarray are shown in Table 1. The majority (26/33) of enrolled patients were female, and all but one sample analyzed by microarray were from females. The recurrent incisional hernia (RH) and normal control (NC) groups analyzed by microarray were comparable (p>0.05) on all demographics except diabetes (p=0.03) and previous surgery (p=0.01), neither of which is unexpected in these populations.

TABLE 1
Demographics of enrolled patients and the subset analyzed by microarray

| | Patients enrolled | | | Patients analyzed by microarray | | |
|---|---|---|---|---|---|---|
| Characteristics | RH (n = 18) | NC (n = 15) | p | RH (n = 9) | NC (n = 8) | p |
| Sex (M/F) | 4/14 | 3/12 | 0.99 | 0/9 | 1/7 | 0.47 |
| Age | 553.2 | 44.9 | 0.14 | 50.9 | 39.1 | 0.23 |
| BMI | 36.6 | 30.5 | 0.03 | 39.2 | 31.4 | 0.10 |
| Smoker | 8 | 2 | 0.07 | 4 | 2 | 0.62 |
| Diabetes | 7 | 0 | 0.01 | 5 | 0 | 0.03 |
| Previous surgery | 18 | 6 | 0.01 | 9 | 3 | 0.01 |

Identification of differential gene expression in the skin and fascia of recurrent incisional hernia patients via microarray

Illumina microarray data revealed that 142 complete genes and 25 expressed sequence tags (ESTs) for a total of 167 genes were differentially expressed in the skin, and 6 complete genes and 1 EST were differentially expressed in fascia for a total of 7 genes. While the full results are included in Online Resources 1 and 2, a representative list of genes is reported in Tables 2 and 3. These were selected based on our interest in hernia formation and wound healing, as well as regulation of transcription and immunology.

TABLE 2
Selected genes from skin of recurrent incisional hernia patients significantly over- or under
expressed in comparison with skin from NC, in ascending order of fold change (NC/RH )

| Gene | Fold | |
|---|---|---|
| symbol | change | Gene name |
| GREM1 | 0.49 | Gremlin 1, cysteine knot superfamily, homolog (Xenopus laevis) |
| TP63 | 0.5 | Tumor protein p63 |
| KRT15 | 0.53 | Keratin 15 |
| TFAP2C | 0.59 | Transcription factor AP-2 gamma (activating enhancer binding |
| | | protein 2 gamma) |
| KLF5 | 0.63 | Kruppel-like factor 5 (intestinal) |
| ELL2 | 0.66 | Elongation factor, RNA polymerase II, 2 |
| NAP1L1 | 0.66 | Nucleosome assembly protein 1-like 1 |
| COL5A2 | 1.51 | Collagen, type V, alpha 2 |
| PDXK | 1.51 | Pyridoxal (pyridoxine, vitamin B6) kinase |
| GHR | 1.54 | Growth hormone receptor |
| NUCB1 | 1.55 | Nucleobindin 1 |
| CD81 | 1.56 | CD81 molecule |
| RBPMS2 | 1.59 | RNA binding protein with multiple splicing 2 |
| TIMP1 | 1.59 | TIMP metallopeptidase inhibitor 1 |
| ANXA5 | 1.59 | Annexin A5 |
| CAV1 | 1.60 | Caveolin 1, caveolae protein, 22 kDa |
| THY1 | 1.62 | Thy-1 cell surface antigen |
| PMP22 | 1.62 | Peripheral myelin protein 22 |
| COL5A1 | 1.63 | Collagen, type V, alpha 1 |
| FBLN1 | 1.63 | Fibulin 1 |
| FBN1 | 1.63 | Fibrillin 1 |
| CLDN5 | 1.66 | Claudin 5 (transmembrane protein deleted in velocardiofacial |
| | | syndrome) |
| MSX1 | 1.66 | Msh homeobox 1 |
| COL1A2 | 1.69 | Collagen, type I, alpha 2 |
| PDGFRB | 1.7 | Platelet-derived growth factor receptor, beta polypeptide |
| FAP | 1.74 | Fibroblast activation protein, alpha |
| DCN | 1.74 | Decorin |
| MCAM | 1.79 | Melanoma cell adhesion molecule |
| COL6A3 | 1.8 | Collagen, type VI, alpha 3 |
| CILP | 1.99 | Cartilage intermediate layer protein, nucleotide |
| | | pyrophosphohydrolase |
| LUM | 2.03 | Lumican |
| COL1A1 | 2.05 | Collagen, type I, alpha 1 |
| FZD4 | 2.11 | Frizzled homolog 4 (Drosophila) |
| CTHRC1 | 2.17 | Collagen triple helix repeat containing 1 |
| HSPB6 | 2.17 | Heat shock protein, alpha-crystallin-related, B6 |
| RBP4 | 2.17 | Retinol binding protein 4, plasma |
| COL3A1 | 2.3 | Collagen, type III, alpha 1 |
| COL4A1 | 2.43 | Collagen, type IV, alpha 1 |
| ANGPTL2 | 2.7 | Angiopoietin-like 2 |
| CD36 | 3.07 | CD36 molecule (thrombospondin receptor) |
| FSTL1 | 3.15 | Follistatin-like 1 |
| PCOLCE2 | 3.64 | Procollagen C-endopeptidase enhancer 2 |
| LEP | 5.03 | Leptin |

TABLE 3
Selected genes from fascia of recurrent incisional hernia patients
over- or under expressed in comparison with fascia from
NC patients in ascending order of fold change (NC/RH)

| Gene symbol | Fold change | Gene name |
|---|---|---|
| GREM1 | 0.23 | Gremlin 1 |
| PRLR | 0.39 | Prolactin receptor |
| LEFTY | 0.43 | Left-right determination factor |
| SCRG1 | 0.44 | Scrapie responsive protein 1 |
| RNF144A | 0.49 | Ring finger protein 1 |
| PDZRN4 | 0.54 | PDZ domain containing ring finger 4 |

Eight discovered genes were directly involved in collagen synthesis (PCOLCE2, CTHRC1, COL1A1, COL3A1, COL4A1, COL5A1, COL5A2, and COL6A3). Moreover, as supported by the literature, several have been associated with hernia formation, Ehlers-Danlos syndrome, and Marfan's syndrome (e.g., COL1A1, COL3A1, COL5A1, FBN1, and TIMP1).

A novel and unexpected gene found to be statistically significant in both the skin and fascia was GREMLIN1 (GREM1, also known as cysteine knot superfamily 1, BMP Antagonist 1, CKTSF1B1; induced in high glucose 2, IHG-2; and down regulated by v-mos, DRM) [19]. In fascia, GREM1 had a fold change of 0.23 (q=0.095, p<10-4), while in skin, it was found to have a fold change of 0.49 (q=0.0009, p<10-7). GREM1 was under expressed in both the skin and fascia of recurrent incisional hernia patients in comparison with NC.

### Gene Ontology Analysis of Differentially Expressed Genes

Gene ontology analyses were performed to determine whether there were common functions or descriptive terms that were statistically abundant in the list of differentially expressed genes, as quantified by odds ratios. Although the fascia gene list was too sparse for analysis, in skin we found more than 53 biologic process (BP) enriched terms, 18 enriched molecular function (MF) terms, and 10 cellular component (CC) terms (Online Resources 3, 4, and 5).

Table 4 represents a sample of important biologic processes that we found to be differentially enriched in skin. For example, in the skin of recurrent incisional hernia patients, many differentially expressed genes were found to be more abundant than expected in biologic processes such as: response to wounding; regulation of immune response; activation of plasma proteins during acute inflammatory response; lipid metabolic process; multicellular organismal development; and cell adhesion. Moreover, these analyses illustrate that many genes such as the collagen genes have diverse functions and appear in several BP categories. For instance, COL3A1 and FBN1 were associated with response to wound healing, blood coagulation, regulation of body fluids, as well as organ development. COL3A1 was also associated with regulation of immune response, regulation of multicellular organismal process, negative regulation of response to stimulus, cell-matrix adhesion, and negative regulation of immune system process.

TABLE 4
Selected results from GO analysis of biologic processes in list of differentially
expressed genes from skin samples

| GO ID | OR | p | Term | Differentially expressed genes in term |
|---|---|---|---|---|
| 0002541 | 7.01 | 0.039 | Activation of plasma proteins | CFD, CFH |
| | | | involved in acute | |
| | | | inflammatory response | |
| 0007160 | 6.62 | 0.001 | Cell-matrix adhesion | COL3A1, ECM2, NID1, EPDR1, THY1 |
| 0050776 | 4.89 | 0.012 | Regulation of immune | COL3A1, CFD, CFH, THY1 |
| | | | response | |
| 0009611 | 3.18 | 0.001 | Response to wounding | COL3A1, CFD, FABP4, FBN1, CFH, |
| | | | | ANXA5, PROK2, VWF, CAV1, AOC3, |
| | | | | CD36 |
| 0007155 | 2.36 | 0.007 | Cell adhesion | FERMT2, COL5A1, COL6A3, VCAN, |
| | | | | DPT, ISLR, LAMA4, MCAM, MFAP4, |
| | | | | S100A4, CLDN5, AOC3, CD36 |

These terms are more abundant than expected and are sorted by odds ratio (OR)

Validation of Gene Expression by qPCR and PCR Array

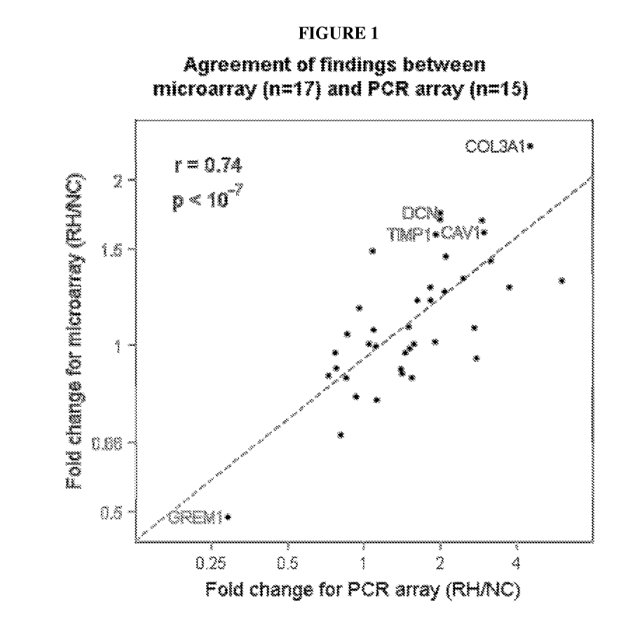

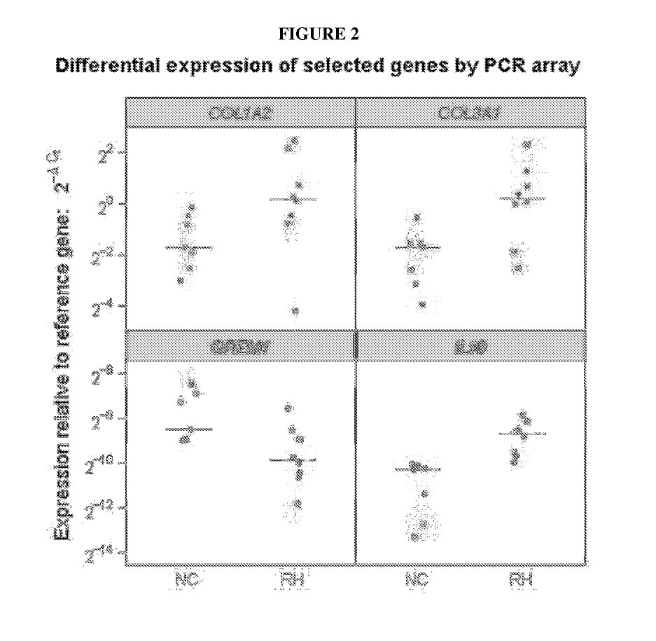

Based upon the Illumina microarray results, COL1A1 and GREM1 were selected for validation by qPCR. COL1A1 was overexpressed (2.33 fold) in the skin of recurrent incisional hernia patients as compared to NC, but was under expressed (0.34 fold) in the fascia. GREM1 was under expressed in both the skin (2.6 fold) and fascia (11.2 fold) of recurrent incisional hernia patients in comparison with NC (Online Resource 6). In order to explore the relationship between other relevant wound-healing genes, such as COL1A1 and COL3A1, a PCR array was used to measure gene expression on a subset of 15 remaining patient samples. Eighty genes on the PCR array were reliably expressed and were analyzed for differences. The PCR array results confirmed the microarray data as illustrated by the strong agreement of fold change (Pearson r=0.74, p<10-7) among the 39 genes common to both arrays which were detectable (FIG. 1). The 22 genes with large fold changes found by PCR array are reported in Table 5, with results for all genes on the PCR array presented in Online Resource 7. The distributions of patient expression levels from the PCR array for four selected differentially expressed genes overlap less than 50% on average (FIG. 2).

TABLE 5

| Gene symbol | Fold change | p |
|---|---|---|
| GREM1* | 0.29 | 0.007 |
| AGT | 2.08 | 0.184 |
| THBS2 | 2.11 | 0.081 |
| TIMP2 | 2.29 | 0.059 |
| HGF | 2.32 | 0.084 |
| ENG | 2.37 | 0.060 |
| MMP2 | 2.47 | 0.054 |
| MMP9* | 2.57 | 0.037 |
| CTGF* | 2.65 | 0.025 |
| ITGB3* | 2.72 | 0.036 |
| MMP3 | 2.78 | 0.063 |
| SMAD6* | 2.78 | 0.033 |
| COL1A2* | 2.92 | 0.035 |
| CAV1* | 2.98 | 0.020 |
| CCL3* | 3.01 | 0.020 |
| THBS1* | 3.15 | 0.012 |
| SERPINE1* | 3.23 | 0.013 |
| ITGA1* | 3.34 | 0.010 |
| LOX* | 3.76 | 0.006 |
| COL3A1* | 4.54 | 0.002 |
| IL10* | 4.83 | 0.001 |
| TIMP4* | 6.02 | 0.001 |

Genes sorted by fold change (RH/NC) in skin by PCR array with fold changes >2 or <0.5 between RH (n = 8) and NC (n = 7), where * denotes p < 0.05

### COL1/COL3 Ratio by Microarray, PCR Array, and Immunohistochemistry

By microarray, COL1A1/COL3A1 ratio in skin of was slightly lower than NC patients, but was not significant (1.33 vs. 1.46, p=0.65). Similar but significant results were found for COL1A2/COL3A1 (0.59 vs. 0.79, p=0.02). Neither of these ratios were statistically different in the fascia. Immunohistochemistry on five patients demonstrated slightly greater staining intensity of COL3A1 than COL1A1 in the skin and fascia from RH patients in comparison with NC. Analysis by PCR array revealed that gene expression of COL3A1 was greater than COL1A2 (the second alpha chain of the collagen 1 molecule) in skin in both groups. According to the manufacturer, COL1A2 was selected because it was referenced more often in relation to fibrosis in public databases than COL1A1. Moreover, the ratio of COL1A2/COL3A1 was decreased in the RH group as compared to NC (1.51 vs. 2.26, p=0.058, one-sided t test). These results agree with reports in the literature [4-12].

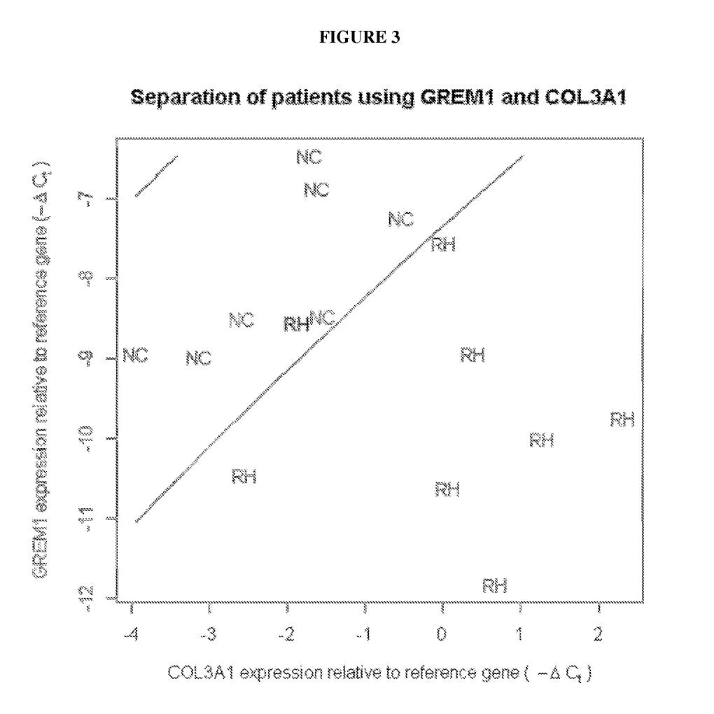

The gene expression ratio of COL1A2/COL3A1, in conjunction with GREM1, was explored as a means of stratifying patients into NC or RH. We also considered COL1A2 and COL3A1 on their own (i.e., not in ratio form) in combination with GREM1. All pairwise combinations of these four markers were considered as means of classifying patients into their correct group (RH or NC) using quadratic discriminant analysis (QDA). QDA may be thought of as a method that yields the best curve (“separation boundary”) that can be drawn in order to maximize the separation between the group means. We found that by using leave-one-out cross-validation, the combination of {GREM1, COL3A1} (FIG. 3) achieved the highest accuracy (86%), followed by either {COL3A1, COL1A2} or {COL1A2, COL1A2/COL3A1} at 73% accuracy, and {GREM1, COL1A2} or {GREM1, COL1A2/COL3A1} at 66% accuracy.

The molecular biology of hernia repair is largely unknown. Equally unclear is why incisional hernia repairs, either laparoscopic or open, frequently recur. We designed a pilot study, using microarrays, to identify potentially specific gene profiles in patients with recurrent incisional hernias (RH). We analyzed the skin and fascia from these patients and compared them to skin and fascia taken from patients who had no history of hernias (NC).

Our study was unique both in using a genomic-based approach (microarray and PCR array) and in taking skin and fascia samples away from the site of the incisional hernia. The acquisition of skin and fascia at the start of the procedure, prior to trocar placement, allowed us to avoid the confounder of biologic and pathologic processes occurring in the hernia (e.g., inflammation, wound healing) that could skew our results. Wound infection, for instance, has been widely reported as the most significant independent prognostic factor for incisional hernia [1, 20-22]. Although technical factors such as type of repair or use of mesh have been attributed to cause recurrence, they do not explain all hernia recurrences [1]. We theorized that variations in gene expression may play a role in wound healing and recurrence.

Our experiments have shown distinct gene expression profiles between the skin and fascia of RH and NC patients. When comparing active gene expression profiles, we found more statistically significant genes in the skin than the fascia. We found greater variability in gene expression in fascia than skin in our samples, which is apparent graphically (Online Resources 8 and 9). Since an increase in variance reduces the power to detect differences, this is the most obvious explanation for the shorter fascia gene list. The functions of the genes in the skin were diverse and included wound healing, transcription regulation, and immunology.

The sparse number of genes in the fascia precluded GO analysis. In the skin, GO analysis further expanded these to 53 BP functions, including regulation of the immune and inflammatory responses, organ development, and cell adhesion. GO analysis also revealed 10 CC and 18 MF categories, with most genes associated with the extracellular region and plasma membrane, and enzyme inhibitor activity and receptor binding, respectively. The relationship of these genes to known biologic functions can assist in our understanding of the basic science of hernia formation.

One of our most intriguing findings was altered GREM1 expression in the skin and fascia of RH patients. Originally isolated from the neural crest of the Xenopus as a bone morphogenetic protein (BMP) antagonist, GREM 1 is an important regulator of limb development and may play a role in regulating organogenesis, body patterning, as well as tissue differentiation [19, 23, 24]. High levels have been found in nondividing and terminally differentiated cells such as neurons, alveolar epithelial cells, and goblet cells [19, 24]. An earlier name of GREM1 was IHG-2 because its expression in glomerular mesangial cells was induced by high glucose, mechanical strain, and TGF-β [25]. GREM1 has been suggested to be a modulator of mesangial cell proliferation and epithelial-mesenchymal transdifferentiation in diabetes and has been shown to have increased expression in various diabetic nephropathy models as well as being involved in the pathophysiology of progressive renal fibrogenetic diseases [26, 27]. Moreover, gene and protein expression have been reported in fibroblast cultures harvested from patients diagnosed with systemic sclerosis [28].

Although GREM1 has not been associated with hernia formation or wound healing, it has been found in the stromal cells of basal cell carcinomas [29]. This group also reported a concomitant expression of FOLLISTATIN (FST) in the stromal cells of basal cell carcinomas [29]. Interestingly, our data showed that FST-like 1 expression accompanied GREM1 expression in the skin of recurrent incisional hernia patients. The findings in the literature support a role for GREM1 in fibrosis of the skin and kidney and are suggestive of a role in hernia formation. The potential role of GREM1 becomes further substantiated when viewed from a perspective that defects in normal wound healing and mechanical strain are frequently cited as causes of hernia formation and recurrence. Although our microarray data were validated by qPCR and PCR array, we are in the process of further testing the role of GREM1 in an expanded population of patients.

More conventional genes of interest from our study were the eight genes directly involved with collagen synthesis and those associated with hernia formation, Ehlers-Danlos syndrome, and Marfan's syndrome such as FBN1. Our data on COL1A1 and COL3A1 were validated by qPCR and PCR array. The ratio of collagen Ito collagen III decreased in the RH patients in comparison with NC as would be expected according to the literature [4-8]. These data are strengthened by the fact that a decrease was seen regardless of which collagen 1 alpha chain was analyzed. The clinical manifestations of Marfan's suggest that alterations in connective tissue stability may play an important role. Mutations in FBN1 are known to cause Marfan's syndrome and have been associated with tissue stability [30]. Recently, an immunohistochemcial study was performed on scar and nonscar regions of human skin and fascia [30]. The authors studied 22 patients who underwent repeated laparotomy: 12 had developed incisional hernia and 10 did not and were used as control. They found that FBN1 may be an important contributing factor to tissue stability and incisional hernia formation [30].

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description, which do not depart from the spirit or scope of the present inventive discovery. Such modifications are also intended to fall within the scope of the appended claims.

All references cited in this specification are hereby incorporated by reference. The discussion of the references herein is intended merely to summarize the assertions made by their authors and no admission is made that any reference constitutes prior art relevant to patentability. Applicant reserves the right to challenge the accuracy and pertinency of the cited references.

### REFERENCES

1. Millikan K W (2003) Incisional hernia repair. Surg Clin N Am 83:1223-1234
2. Awad Z T, Puri V, LeBlanc K, Stoppa R, Fitzgibbons R J Jr, Iqbal A, Filipi C J (2005) Mechanisms of ventral hernia recurrence after mesh repair and a new proposed classification. J Am Coll Surg 201:132-140
3. Franz MG (2006) The biology of hernias and the abdominal wall. Hernia 10:462-471
4. Klinge U, Zheng H, Si Z Y, Schumpelick V, Bhardwaj R, Klosterecurrent incisional hernia alfen B (1999) Synthesis of type I and III collagen, expression of fibronectin and matrix metalloproteinases-1 and -13 in hernial sac of patients with inguinal hernia. Int J Surg Investig 1:219-227
5. Klinge U, Zheng H, Si Z, Schumpelick V, Bhardwaj R S, Muys L, Klosterecurrent incisional hernia alfen B (1999) Expression of the extracellular matrix proteins collagen I, collagen III and fibronectin and matrix metalloproteinase-1 and -13 in the skin of patients with inguinal hernia. Eur Surg Res 31:480-490
6. Klinge U, Si Z Y, Zheng H, Schumpelick V, Bhardwaj R S, Klosterecurrent incisional hernia alfen B (2000) Abnormal collagen I to III distribution in the skin of patients with incisional hernia. Eur Surg Res 32:43-48
7. Rosch R, Junge K, Knops M, Lynen P, Klinge U, Schumpelick V (2003) Analysis of collagen-interacting proteins in patients with incisional hernias. Langenbecks Arch Surg 387:427-43
8. White B, Osier C, Gletsu N, Jeansonne L, Baghai M, Sherman M, Smith C D, Ramshaw B, Lin E (2007) Abnormal primary tissue collagen composition in the skin of recurrent incisional hernia patients. Am Surg 73:1254-1258
9. Liem M S, van der Graaf Y, Beemer F A, van Vroonhoven T J (1997) Increased risk for inguinal hernia in patients with Ehlers-Danlos syndrome. Surgery 122:114-115
10. Rowe D W, Shapiro J R, Poirier M, Schlesinger S (1985) Diminished type I collagen synthesis and reduced alpha 1(I) collagen messenger RNA in cultured fibroblasts from patients with dominantly inherited (type I) osteogenesis imperfecta. J Clin Invest 76:604-611
11. Deak S B, Ricotta J J, Mariani T J, Deak S T, Zatina M A, Mackenzie J W, Boyd CD (1992) Abnormalities in the biosynthesis of type III procollagen in cultured skin fibroblasts from two patients with multiple aneurysms. Matrix 12:92-100
12. Heath J D, Cox D, Goralski T, Retief J (2007) NuGen's WT-Ovation FFPE System coupled with a novel labeling protocol enables genome-wide gene expression profiling of FFPE sample on the Illumina BeadArray platform. Application Note #2. NuGen Technologies
13. Calaluce R, Gubin M M, Davis J W, Magee J D, Chen J, Kuwano Y, Gorospe M, Atasoy U (2010) The RNA binding protein HuR differentially regulates unique subsets of mRNAs in estrogen receptor negative and estrogen receptor positive breast cancer. BMC Cancer 10:126-140
14. Smyth G K (2004) Linear models and empirical bayes methods for assessing differential expression in microarray experiments. Stat Appl Genet Mol Biol 3:Article3
15. Ramos-Vara J A, Beissenherz M E (2000) Optimization of immunohistochemical methods using two different antigen retrieval methods on formalin-fixed paraffin-embedded tissues: experience with 63 markers. J Vet Diagn Invest 12:307-311
16. Gubin M M, Calaluce R, Davis J W, Magee J D, Strouse C S, Shaw D P, Ma L, Brown A, Hoffman T, Rold T L, Atasoy U (2010) Overexpression of the RNA binding protein HuR impairs tumor growth in triple negative breast cancer associated with deficient angiogenesis. Cell Cycle 9:3337-3346
17. Pelch K E, Schroder A L, Kimball P A, Sharpe-Timms K L, Davis J W, Nagel S C (2010) Aberrant gene expression profile in a mouse model of endometriosis minors that observed in women. Fertil Steril 93:1615-1627, e1817
18. Benjamini Y, Hochberg Y (1995) Controlling the false discovery rate: a practical and powerful approach to multiple testing. J R Stat Soc B 57:289-300
19. Avsian-Kretchmer O, Hsueh A J (2004) Comparative genomic analysis of the eight-membered ring cystine knot-containing bone morphogenetic protein antagonists. Mol Endocrinol 18:1-12
20. Bucknall T E, Cox P J, Ellis H (1982) Burst abdomen and incisional hernia: a prospective study of 1129 major laparotomies. Br Med J (Clin Res Ed) 284:931-933
21. Greenall M J, Evans M, Pollock A V (1980) Midline or transverse laparotomy? A random controlled clinical trial. Part I: Influence on healing. Br J Surg 67:188-190
22. Gys T, Hubens A (1989) A prospective comparative clinical study between monofilament absorbable and non-absorbable sutures for abdominal wall closure. Acta Chir Belg 89:265-270
23. Hsu D R, Economides A N, Wang X, Eimon P M, Harland R M (1998) The Xenopus dorsalizing factor Gremlin identifies a novel family of secreted proteins that antagonize BMP activities. Mol Cell 1:673-683
24. Topol L Z, Modi W S, Koochekpour S, Blair D G (2000) DRM/GREMLIN (CKTSF1B1) maps to human chromosome 15 and is highly expressed in adult and fetal brain. Cytogenet Cell Genet 89:79-84
25. McMahon R, Murphy M, Clarkson M, Taal M, Mackenzie H S, Godson C, Martin F, Brady HR (2000) IHG-2, a mesangial cell gene induced by high glucose, is human gremlin. Regulation by extracellular glucose concentration, cyclic mechanical strain, and transforming growth factor-beta1. J Biol Chem 275:9901-9904
26. Lappin D W, McMahon R, Murphy M, Brady H R (2002) Gremlin: an example of the re-emergence of developmental programmes in diabetic nephropathy. Nephrol Dial Transpl 17(Suppl 9):65-67
27. Murphy M, McMahon R, Lappin D W, Brady H R (2002) Gremlins: is this what renal fibrogenesis has come to? Exp Nephrol 10:241-244
28. Meyringer R, Neumann E, Judex M, Landthaler M, Kullmann F, Scholmerich J, Gay S, Tamer I H, Distler O, Muller-Ladner U (2007) Analysis of gene expression patterns in systemic sclerosis fibroblasts using RNA arbitrarily primed-polymerase chain reaction for differential display. J Recurrent incisional hernia eumatol 34:747-753
29. Sneddon J B, Zhen H H, Montgomery K, van de Rijn M, Tward A D, West R, Gladstone H, Chang H Y, Morganroth G S, Oro A E et al (2006) Bone morphogenetic protein antagonist gremlin 1 is widely expressed by cancer-associated stromal cells and can promote tumor cell proliferation. Proc Natl Acad Sci USA 103:14842-14847
30. Fricke M, Langer C, Brunner E, Sakai L Y, Fuzesi L, Reinhardt D P, Quondamatteo F (2008) Fibrillin-1 in incisional hernias: an immunohistochemical study in scar and non-scar regions of human skin and muscle fasciae. J Anat 212:674-685
31. Llaguna O H, Avgerinos D V, Nagda P, Elfant D, Leitman I M, Goodman E. Does prophylactic biologic mesh placement protect against the development of incisional hernia in high-risk patients? World J. Surg. 2011 July; 35(7):1651-5.
32. Kissane N A, Itani K M. A decade of ventral incisional hernia repairs with biologic acellular dermal matrix: what have we learned? Plast Reconstr Surg. 2012 November; 130(5 Suppl 2):194S-202S.
33. Aura T, Habib E, Mekkaoui M, Brassier D, Elhadad A. Laparoscopic tension-free repair of anterior abdominal wall incisional and ventral hernias with an intraperitoneal Gore-Tex mesh: prospective study and review of the literature. J Laparoendosc Adv Surg Tech A. 2002 August; 12(4):263-7.
34. Helgstrand F, Gögenur I, Rosenberg J. Prevention of parastomal hernia by the placement of a mesh at the primary operation. Hernia. 2008 December; 12(6):577-82.
35. Martorana G, Carlucci M, Alia C, Barrianco G, Iacopinelli S M, Labruzzo C, Noto N, Restivo F P, Viola M, Mastrandrea G. [Laparoscopic incisional hernia repair: our experience and review of the literature]. Chir Ital. 2007 September-October; 59(5):671-7.

What is claimed is:

1. A method of treating an abdominal surgical incision in a subject in need thereof comprising the steps of:
a. measuring the level of expression of a gene or combination of genes selected from the group consisting of COL1A2, COL3A1, GREM1, and IL10 in a tissue sample obtained from the subject;
b. comparing the level of expression of the gene or combination of genes in the tissue sample to that in a control sample;
c. determining a risk level for an incisional hernia in the subject following surgery, wherein the subject has a high risk for an incisional hernia if the level of expression of the gene or combination of genes in the subject's tissue sample displays a greater than 1.5 fold change compared to the level of expression of the gene or combination of genes in the control sample, and a normal risk for an incisional hernia if the level of expression of the gene or combination of genes in the subject's tissue sample displays a 1.5 fold change or less compared to the level of expression of the gene or combination of genes in the control sample; and
d. treating the subject based upon the subject's risk level for an incisional hernia as determined in step c.

2. The method of claim 1, wherein the tissue sample is obtained from the subject's skin, blood, or fascia.

3. The method of claim 1, wherein the gene is GREM1.

4. The method of claim 1, wherein the subject has a high risk for an incisional hernia if the level of expression of GREM1 in the subject's tissue sample is lower than the level of expression of GREM1 in the control sample.

5. The method of claim 1, wherein the subject has a high risk for an incisional hernia if the level of expression of COL1A2, COL3A1, or IL10, or combinations thereof, in the subject's tissue sample is higher than that in the control sample.

6. The method of claim 1, wherein the level of gene expression in a. or b. is measured by microarray, PCR array, or immunohistochemistry.

7. The method of claim 1, wherein the level of gene expression in a. and b. is measured via the quantity of nucleic acid transcripts produced by the gene or combination of genes.

8. The method of claim 1, wherein the gene expression is measured via the quantity of protein expressed from the gene or combination of genes.

9. The method of claim 1, wherein the subject determined to have a high risk for an incisional hernia is treated with placement of surgical mesh in the abdominal incision.

10. The method of claim 9, wherein the surgical mesh is affixed to abdominal tissue bridging the abdominal incision.

11. The method of claim 1, wherein the subject determined to have a high risk for an incisional hernia is treated with laparoscopic surgery.

12. A method of diagnosing a risk level for an incisional hernia in a subject following abdominal surgery, comprising the steps of:
a. measuring the level of expression of a gene or combination of genes selected from the group consisting of COL1A2, COL3A1, GREM1, and IL10 in a tissue sample obtained from the subject;
b. comparing the level of expression of the gene or combination of genes in the tissue sample to that in a control sample;
c. determining a risk level for an incisional hernia in the subject following surgery,
wherein the subject has a high risk for an incisional hernia if the level of expression of the gene or combination of genes in the subject's tissue sample displays a greater than 1.5 fold change compared to the level of expression of the gene or combination of genes in the control sample, and a normal risk for an incisional hernia if the level of expression of the gene or combination of genes in the subject's tissue sample displays a 1.5 fold change or less compared to the level of expression of the gene or combination of genes in the control sample.

13. The method of claim 12, wherein the tissue sample is obtained from the subject's skin, blood, or fascia.

14. The method of claim 12, wherein the gene is GREM1.

15. The method of claim 12, wherein the subject has a high risk for an incisional hernia if the level of expression of GREM1 in the subject's tissue sample is lower than the level of expression of GREM1 in the control sample.

16. The method of claim 12, wherein the subject has a high risk for an incisional hernia if the level of expression of COL1A2, COL3A1, or IL10, or combinations thereof, in the subject's tissue sample is higher than that in the control sample.

17. The method of claim 12, wherein the level of gene expression in a. or b. is measured by microarray, PCR array, or immunohistochemistry.

18. The method of claim 12, wherein the level of gene expression in a. and b. is measured via the quantity of nucleic acid transcripts produced by the gene or combination of genes.

19. The method of claim 12, wherein the gene expression is measured via the quantity of protein expressed from the gene or combination of genes.

20. A product comprising isolated biomarkers bound to a biochip array, wherein the biomarkers are selected from the group consisting of COL1A2, COL3A1, IL10, and combinations thereof.

21. The product of claim 20, wherein the biomarker is GREM1.

22. A product comprising purified biomarkers bound to a microarray comprising addressable locations using a biospecific capture reagent, wherein the biomarkers are a gene or combination of genes selected from the group consisting of COL1A2, COL3A1, GREM1, and IL10.

23. A product of claim 22, wherein the biomarker is GREM1.

24. A kit for diagnosing a risk level for an incisional hernia in a subject following abdominal surgery, comprising:
a. purified biomarkers bound to a microarray comprising addressable locations using an adsorbent or capture reagent, wherein the purified biomarkers are selected from the group consisting of COL1A2, COL3A1, GREM1, IL10, or combinations thereof, and
b. written instructions for diagnosing a risk level for an incisional hernia in a subject following abdominal surgery, comprising the steps of:
i. measuring the level of expression of a gene or combination of genes selected from the group consisting of COL1A2, COL3A1, GREM1, and IL10 in a tissue sample obtained from the subject;
ii. comparing the level of expression of the gene or combination of genes in the tissue sample to that in a control sample;
iii. determining a risk level for an incisional hernia in the subject following surgery,
wherein the subject has a high risk for an incisional hernia if the level of expression of the gene or combination of genes in the subject's tissue sample displays a greater than 1.5 fold change compared to the level of expression of the gene or combination of genes in the control sample, and a normal risk for an incisional hernia if the level of expression of the gene or combination of genes in the subject's tissue sample displays a 1.5 fold change or less compared to the level of expression of the gene or combination of genes in the control sample.

25. The kit of claim 24, wherein the biomarker is GREM1.

26. A biochip array comprising isolated biomarkers, wherein the biomarkers are selected from the group consisting of COL1A2, COL3A1, GREM1, IL10, and combinations thereof.

27. The biochip array of claim 26, wherein the biomarker is GREM1.

28. A biochip array having a plurality of addressable locations, each comprising at least one isolated biomarker, wherein the biomarkers are selected from the group consisting of COL1A2, COL3A1, GREM1, IL10, and combinations thereof.

29. The biochip array of claim 28, wherein the biomarker is GREM1.

30. The biochip array of claim 28, having at least two addressable locations, each comprising at least one isolated biomarker, wherein the two or more biomarkers are selected from the group consisting of COL1A2, COL3A1, GREM1, IL10, and combinations thereof.

31. The biochip array of claim 28, having at least three addressable locations, each comprising at least one isolated biomarker, wherein the three or more biomarkers are selected from the group consisting of COL1A2, COL3A1, GREM1, IL10, and combinations thereof.

32. The biochip array of claim 28, having at least four addressable locations, each comprising at least one isolated biomarker, wherein the four biomarkers are COL1A2, COL3A1, GREM1, and IL10.

33. A product comprising at least one isolated biomarker bound to a bead by a biospecific capture reagent, wherein the biomarkers are selected from the group consisting of COL1A2, COL3A1, GREM1, IL10, and combinations thereof.

34. The product of claim 33, wherein one biomarker is bound to the bead.

35. The product of claim 34, wherein the biomarker is GREM1.

36. The product of claim 35, wherein the biospecific capture reagent is an antibody.

37. The product of claim 34, comprising a plurality of beads of at least one bead type, wherein each bead type comprises an isolated biomarker bound to the bead by a biospecific capture reagent, and wherein the biomarkers are selected from the group consisting of COL1A2, COL3A1, GREM1, and IL10.

38. The product of claim 37, wherein the biospecific capture reagent is an antibody.

39. The product of claim 37, comprising at least two bead types.

40. The product of claim 37, comprising at least three bead types.

41. The product of claim 37, comprising at least four bead types.

42. The product of claim 30, comprising at least two isolated biomarkers bound to a bead by a biospecific capture reagent, wherein the biomarkers are selected from the group consisting of COL1A2, COL3A1, GREM1, IL10, and combinations thereof.

43. The product of claim 42, wherein the biomarker is GREM1.

44. The product of claim 42, wherein the biospecific capture reagent is an antibody.

45. A biochip comprising one or more isolated biomarkers, wherein the biomarkers are selected from the group consisting of COL1A2, COL3A1, GREM1, IL10, and combinations thereof.

46. The biochip of claim 45, wherein the biomarker is GREM1.

47. The biochip of claim 45, wherein the isolated biomarkers are present at addressable locations.

48. The biochip of claim 47, having at least two addressable locations, each comprising a different isolated biomarker, wherein the biomarkers are selected from the group consisting of COL1A2, COL3A1, GREM1, IL10, and combinations thereof.

49. The biochip of claim 47, having at least three addressable locations, each comprising a different isolated biomarker, wherein the biomarkers are selected from the group consisting of COL1A2, COL3A1, GREM1, IL10, and combinations thereof.

50. The biochip of claim 47, having at least four addressable locations, each comprising a different isolated biomarker, wherein the biomarkers are COL1A2, COL3A1, GREM1, and IL10.
